(19) United States
(12) Patent Application Publication
(10) Pub. No.: US 2007/0087326 A1
(43) Pub. Date: Apr. 19, 2007

(54) Method of measuring the biological activity of an urotensin II receptor

(76) Inventors: Lisa Minor, Flemington, NJ (US); Jenson Qi, Chalfont, PA (US); Yuanping Wang, Dresher, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/502,774

(22) Filed: Aug. 10, 2006

Related U.S. Application Data
(60) Provisional application No. 60/708,220, filed on Aug. 15, 2005.

(51) Int. Cl. C12Q 1/00

(52) U.S. Cl. 435/4

(57) ABSTRACT

Administration of U-II to a cell having a functional urotensin II receptor caused an increase in the electrical impedance of the cell in a receptor specific and dose dependent manner. Thus, the present invention provides methods of measuring the biological activity of an U-II receptor by monitoring the electrical impedance of the cell, and the use of the methods to identify a cell having a functional U-II receptor, as well as to identify compounds that increase or decrease the biological activity of an U-II receptor.

### CROSS REFERENCE TO RELATED APPLICATIONS

This application claims priority to Application No. 60/708,220 filed on Aug. 15, 2005, the entire contents of which are incorporated by reference herein.

### FIELD OF THE INVENTION

The present invention relates to methods of measuring the biological activity of an urotensin receptor. Particularly the present invention relates to methods of monitoring the biological activity of an urotensin receptor by measuring the electrical impedance of a cell and uses of the methods.

### BACKGROUND OF THE INVENTION

Urotensin-II (U-II) is a vasoactive, somatosatin-like cyclic peptide (Coulouarn et al., 1999, FEBS Lett 457(1): 28-32). U-II was originally isolated from the teleost urophysis, and was shown to be involved in the cardiovascular regulation, osmoregulation, and regulation of lipid metabolism in fish (Ohsaka et al., 1986, J. Neurosci 6:2730-2735; and Conlon et al., 1996, J. Exp. Zool. 275:226-238). The genes encoding orthologs of U-II precursor proteins have since been cloned from various species, for example, rat (Marchese et al., 1995, Genomics 29: 335-344), human (Coulouarn et al., 1998, Proc. Natl. Acad. Sci. USA 95: 15803-15808; and Ames et al., 1999, Nature 401(6750): 282-6), and mouse (Coulouarn et al., 1999, supra). Human U-II is found within both vascular and cardiac tissue (including coronary atheroma). In addition, U-II immunoreactivity is also found within central nervous system and endocrine tissues (Ames et al., supra).

G-protein-coupled receptor 14 (GPR14), also known as sensory epithelium neuropeptide-like receptor (SENR), was recently identified as to function as an U-II receptor (Ames et al., supra). GPR14 was cloned as an orphan receptor with similarity to members of the somatostatin/opioid family. Human U-II binds to recombinant human GPR14 with high affinity and the binding is functionally coupled to calcium mobilization. The receptor of U-II (UT receptor) has also been identified and characterized from other animals, for example, mouse and monkey (Elshourbagy et al., 2002, Br. J. Pharmacol. 36: 9-22). The UT receptor is expressed abundantly in the spinal cord, and also in heart, lungs, blood vessels, kidney, and brain (Russell, 2004, Pharmcology & Therapeutics 103: 223-243).

Studies have demonstrated that U-II is both an endothelium independent vasoconstrictor (Ames et al., supra; Maguire et al., 2000, Br. J. Pharmacol. 131(3): 441-6) and an endothelium dependent vasodilator (Bottrill, 2000; Br. J. Pharmacol. 130(8): 1865-70; Zhang et al., 2003, Am. J. Physiol. Renal. Physiol., 285, F792-8). Emerging roles of U-II in cardiovascular diseases have been implicated (Russell supra). Recent evidence suggests that the UT receptor system is up-regulated in multi-organ disease states, such as congestive heart failure (CHF), pulmonary hypertension, and chronic renal failure. A number of non-peptide UT receptor antagonists have been developed with the aim of dampening harmful effects of over-activated UT receptors (see, i.e., Douglas et al, 2004, Trends. Pharmacol. Sci. 25: 76-85). However, U-II exhibits significant species differences, as well as regional and functional differences between vessels (Douglas et al., 2000, Br. J. Pharmacol. 131(7): 1262-74). Molecules identified as antagonist for the rat receptor can behave as agonists against the monkey receptor (Behm, et al., 2004, European Journal of Pharmacology, 492(2-3): 113-116). Thus, it is critical to confirm the effect of a putative drug-like molecule on the biological activities of an endogeneous human UT receptor in a cellular functional assay.

Until recently, there lacked a suitable model cellular system for studying the biological activity of an endogeneous U-II receptor. Qi reported that primary human skeletal muscle myoblasts bind U-II (Qi, et al., 2005, Peptides 26(4): 683-690). Douglas et al. screened a large and diverse collection of primate and rodent cell lines (49 in total) for the presence of readily detectable levels of specific U-II binding sites using a crude whole-cell screening approach (Douglas et al., 2004, Br. J. Pharmacol. 142(6): 921-32). Out of the 49 screened, only 3 cell lines exhibited a significant binding signal. The three cell lines are SJRH30 (ATCC® Number: CRL-2061™, also named RC13, or RMS13), TE671, and a rat medullary thyroid cell line (6-23). Both TE671 and rat medullary thyroid cells line (6-23) displayed poor U-II binding site densities (about 5-10% of that recorded in SJRH30).

The biological activity of an endogeneous U-II receptor has been measured as calcium mobilization in only very few cell lines. Qi reported a slight though appreciable calcium mobilization in response to U-II in primary human skeletal muscle myoblasts (Qi, et al., 2005 supra). Douglas et al. (2004, supra) observed that “only ˜10% SJRH30 cells exposed to hU-II responded with an appreciable [Ca²⁺]ᵢ response,” and “the magnitude of the hU-II-induced [Ca²⁺]ᵢ varied significantly between individual cells from ˜10 nM to several hundred nM over baseline.” Most recently, sub-clones of SJRH30, for example 6D9, have been isolated that have increased U-II binding sites and more robust calcium mobilization response (Minor et al., 2005, U.S. Application Ser. No. 60/708,221, filed Aug. 15, 2005).

A label-free cell-based cellular dielectric spectroscopy (CDS) technology has recently been applied to assess pharmacological activities of cell surface receptors, including the G-protein-coupled receptors (GPCRs) (WO 2005005979). However, it was uncertain prior to this invention whether the CDS technology could be used to specifically detect the biological activity of an endogenous urotensin receptor in a cell because of the lack of or weak U-II binding or U-II stimulated calcium mobilization response that could be measured from previous studies.

To facilitate the development of new compounds that regulate the biological activity of UT receptor, there is a need to establish a cellular functional assay that allows robust and simple measurement of the ability of a candidate compound to increase or decrease the biological activity of an UT receptor, endogeneous or recombinantly expressed in the cell.

### SUMMARY OF THE INVENTION

It is now discovered that administration of U-II to a cell having a functional urotensin II receptor caused an increase in the electrical impedance of the cell in a receptor specific and dose dependent manner.

Thus, a general aspect of the invention is a functional assay of the biological activity of an urotensin receptor in a cell comprising the step of measuring the electrical impedance of the cell. The invention provides methods of using the functional assay to identify cells having a functional U-II receptor, and compounds that increases or decreases the biological activity of an U-II receptor.

In a particular embodiment, the electrical impedance of the cell is measured using a cellular dielectric spectroscopy (CDS) device.

### BRIEF DESCRIPTION OF THE DRAWINGS

FIG. 1 shows cell density-dependence response to Urotensin II (500 nM) as measured by the CDS: A—RMS13 cells, and B—6D9 cells.

FIG. 2 shows that as measured by the CDS, U-II caused a dose dependent increase in the electrical impedance of the cells: square—RMS13 with an EC₅₀ of about 2.7×10⁻¹⁰ M; and triangle—6D9 cells with an EC₅₀ of about 4.2×10⁻¹⁰ M.

FIG. 3 shows that as measured by the CDS, the U-II stimulated increase in the electrical impedance of the cells is specific to the U-II receptor: A—Receptor specificity on RMS13; B—Receptor specificity on 6D9 cells; and C—Receptor specificity on CHOrUII cells.

FIG. 4A shows that as measured by RT-CES™, U-II caused a dose dependent increase in the electrical impedance of the cells. The effect of U-II on impedance was measured in 6D9 cells by the RT-CES™ system. The data were normalized to a time point just prior to agonist addition.

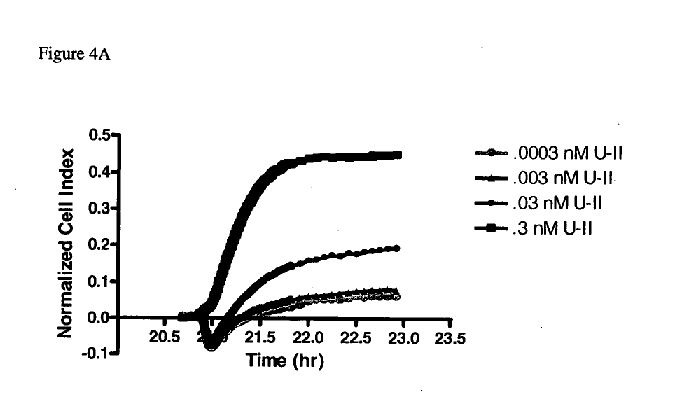

FIG. 4B shows that as measured by RT-CES™, U-II caused a dose dependent increase in the electrical impedance of the cells. The U-II dose response on impedance in 6D9 cells was measured by the RT-CES™ system. The data are presented as raw cell index data (not normalized) and normalized data (normalized to a time point just prior to agonist addition).

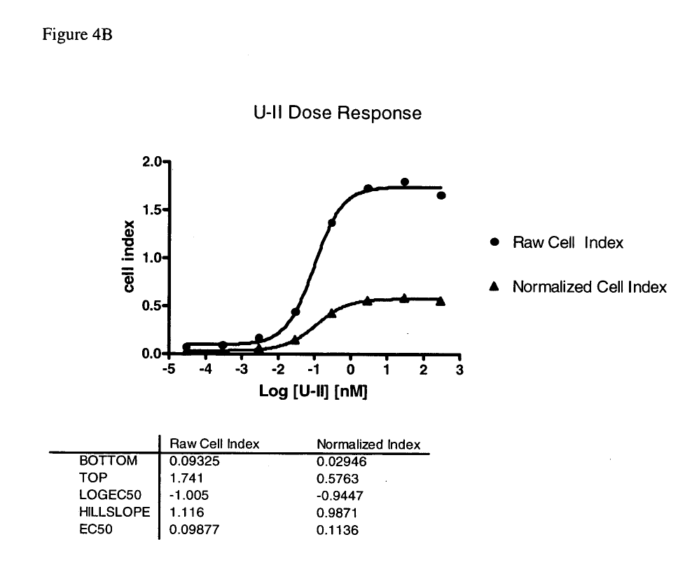

### DETAILED DESCRIPTION OF THE INVENTION

All publications cited hereinafter are hereby incorporated by reference. Unless defined otherwise, all technical and scientific terms used herein have the same meaning as commonly understood to one of ordinary skill in the art to which this invention pertains.

It must be noted that as used herein and in the appended claims, the singular forms “a,” “an,” and “the” include plural reference unless the context clearly dictates otherwise. Thus, for example, a reference to “a cell” is a reference to one or more cells and includes equivalents thereof known to those skilled in the art and so forth.

As used herein, the terms “comprising”, “containing”, “having” and “including” are used in their open, non-limiting sense.

The following are some abbreviations that are at times used in this specification:

ATCC=American Type Culture Collection

CDS=cellular dielectric spectroscopy

GPCR=G protein coupled receptor;

HTS=high throughput screen;

U-II=Urotensin-II;

URP=Urotensin-II-related peptide

UT receptor=Urotensin-II receptor

As used herein, the term “about” means plus or minus 10% of the number to which reference is being made.

As used herein, “calcium mobilization” refers to the process whereby the concentration of intracellular free Ca²⁺, also denoted [Ca²⁺]ᵢ, increases or decreases during signal transduction. [Ca²⁺]ᵢ increases due to, for example, release of Ca²⁺ from internal storage, or increased influx of Ca²⁺ across the plasma membrane and into the cell.

As used herein, a “cell clone” refers to a population of cells derived from a single common ancestral cell by mitosis in eukaryotes, such as a human cell clone, or by binary fission in prokaryotes. Although cells within a cell clone are presumed to be genetically identical, mutational events may abrogate the genetic homogeneity.

As used herein, “sub-clone” is a process whereby a cell clone is obtained from one or few ancestral cells of a parental cell clone. In a particular embodiment, cells within the sub-cloned cell clone have less genetic variability to one to another than cells within the parental cell clone. Depending on the context, “sub-clone” can also refer to the cell clone resulting from the sub-cloning process. To ensure genetic stability, the clone is frozen in early passage and cultures replaced with the frozen stocks at regular intervals.

As used herein, “cellular dielectric spectroscopy” or “CDS” refers to a label-free cell-based technology that measures the impedance of an intact cell to classify and characterize any unique global cellular event such as signal transduction from ligand/receptor interactions, cytotoxicity, apoptosis, tumor cell progression, or stem cell differentiation. Specific examples of CDS and the applications of the CDS are described in WO2005005979. CellKey™, a fully automated CDS system designed for target validation and secondary screening laboratories is commercially available from MDS Sciex (Concord, Ontario).

As used herein, “EC₅₀” refers to the concentration causing 50% enhancement or increase in activity in the system being measured. For example, in the CDS electrical impedance assay, EC₅₀ is defined as that concentration of a compound that causes a 50% increase in the electrical impedance of the cell.

As used herein, the terms “electrical impedance”, “impedance”, and “cellular impedance” used interchangeably herein, each refers to the ratio of voltage to current as described by Ohm's law (Z=V/I). In a particular embodiment of the invention, the “electrical impedance” of a cell can be measured using a CDS system. For example, cells are seeded onto a custom 96-well microplate (CellKey™ standard 96 Well) that contains electrodes at the bottom of the wells. The CellKey™ instrument supplies constant voltage (V) to cells plated on the electrodes producing current that flows around and between cells (extracellular current, I_{ec}) and through cells (transcellular current, I_{tc}). The CellKey™ instrument measures changes in impedance (ΔZ_{ec}, ΔZ_{tc}, and ΔZ=ΔZ_{ec}+ΔZ_{tc}) upon stimulation of different cell surface receptors. Changes in impedance can be caused by physiological changes in the cell, such as changes in cell adherence to their substrate, changes in cell shape and volume, and changes in cell-cell interactions, etc. These physiological changes will affect the flow of extracellular (I_{ec}) and transcellular (I_{tc}) current and hence the magnitude and characteristics of the signal measured. Each of these physiological changes can be linked to receptor stimulation through classical signaling pathways.

As used herein, “urotensin-II”, “U-II”, or “U2”, used interchangeably herein, each refers to a peptide having a conserved cyclic hexapeptide, SEQ ID NO: 1, CFWKYC, wherein an intramolecular disulfide bond is formed between the two cysteines of the hexapeptide. Examples of “urotensin-II” include, but are not limited to, those listed in Table 1, with an intramolecular disulfide bond between two the cysteines of the hexapeptide as listed in SEQ ID NO: 1. “Urotensin-II” also includes the so-called U-II-related peptide (URP), for example, consisting essentially of SEQ ID NO: 10, ACFWKYCV, with an intramolecular disulfide bond formed between the two cysteines (Sugo et al., 2003, Biochem Biophys Res Commun 2003; 310:860-8). An “urotensin-II” can be isolated from a natural source, such as an U-II producing animal. An “urotensin-II” can also be synthesized via any in vitro method, such as an in vitro peptide synthesis reaction.

TABLE 1
Examples of urotensin-II from various species

| | |
|---|---|
| Species | Sequence of urotensin-II |

| | |
|---|---|
| Human | SEQ ID NO: 2, ETPDCFWKYCV |
| Frog | SEQ ID NO: 3, AGNLSECFWKYCV |
| Trout | SEQ ID NO: 4, GGNSECFWKYCV |
| Carp α | SEQ ID NO: 5, GGGAECFWKYCV |
| Porcine-1 | SEQ ID NO: 6, GTPSECFWKYCV |
| Porcine-2 | SEQ ID NO: 7, GPPSECFWKYCV |
| Rat-1 | SEQ ID NO: 8, HGTAPECFWKYCI |
| Mouse | SEQ ID NO: 9, HGAAPECFWKYCI |

A “functional equivalent of urotensin-II” is a chemical entity that has all or part of the biological activity of urotensin-II, i.e., to bind to an urotensin-II receptor, and the binding can be functionally coupled to calcium mobilization. Examples of “functional equivalent of urotensin-II” include, but are not limited to, modifications or truncations of urotensin-II, or fusion proteins comprising urotensin-II, that maintain all or part of the biological activities of an urotensin-II. “Functional equivalent of urotensin-II” also includes, but are not limited, to the non-peptide U-II mimetics, non-peptide UT receptor agonists, inverse agonists or antagonists. The “functional equivalent of urotensin-II” can be from either natural or non-natural sources. Non-natural sources include, for example, recombinant or synthetic sources.

As used herein, an “urotensin II receptor”, “U-II receptor”, “UTR2”, “UT receptor” or “U2R”, used interchangeably, each refers to a G-protein-coupled receptor protein that binds to an urotensin II (U-II) or an analog thereof, and the binding can be functionally coupled to calcium mobilization. An “urotensin II receptor”, can (1) have greater than about 60% amino acid sequence identity to a human U-II receptor (NCBI protein accession number: NP_{—}061822); (2) bind to antibodies, e.g., polyclonal or monoclonal antibodies, raised against a human U-II receptor (NCBI protein accession number: NP_{—}061822); or (3) be encoded by a polynucleotide that specifically hybridizes under stringent hybridization conditions to a nucleic acid molecule having a sequence that has greater than about 60% nucleotide sequence identity to the coding region of a human U-II receptor cDNA (NCBI nucleotide accession number: NM_{—}018949). “Stringent hybridization conditions” has the meaning known in the art, as described in Sambrook et al., Molecular Cloning: A Laboratory Manual, Second Edition, Cold Spring Harbor Laboratory, Cold Spring Harbor, N.Y., (1989). An exemplary stringent hybridization condition comprises hybridization in 6×sodium chloride/sodium citrate (SSC) at about 45° C., followed by one or more washes in 0.2×SSC and 0.1% SDS at 50-65° C.

In some embodiments, the “U-II receptor” has greater than about 65, 70, 75, 80, 85, 90, or 95 percent amino acid sequence identity to a human U-II receptor (NCBI protein accession number: NP_{—}061822). Exemplary U-II receptor includes human U-II receptor, which includes structural and functional polymorphisms of the human U-II receptor depicted in NCBI protein accession number: NP_{—}061822. “Polymorphism” refers to a set of genetic variants at a particular genetic locus among individuals in a population. U-II receptor also includes orthologs of the human U-II receptor in other animals such as rat (i.e., NCBI protein accession NO: NP_{—}065412), mouse (i.e., NCBI protein accession NO: NP_{—}663415), pig, dog and monkey.

An “endogeneous urotensin II binding site” refers to a site on which an urotensin II can specifically bind to, and the site is naturally produced by or associated with the cell. “Naturally” in this context means that the U-II binding site is not recombinantly made, i.e., not genetically altered or modified by artificial means. In one embodiment, an endogeneous urotensin II binding site is found on an endogeneous urotensin II receptor. A “recombinant urotensin II binding site” refers to a site on which an urotensin II can specifically bind to, and the site is recombinantly produced by or associated with the cell. “Recombinantly” in this context means that the U-II binding site is made from a recombinant source, i.e., a source that is genetically altered or modified by artificial means.

The number of the urotensin II binding sites of a cell, whether they are endogeneous or recombinant, can be calculated using any methods known to a person skilled in the art. In one embodiment, the number of urotensin II binding sites of a cell can be calculated from the U-II receptor-ligand binding curve resulting from an U-II receptor-binding assay, see for example, Qi et al. (2005, supra) describes a specific example on how to measure and calculate the number of urotensin II binding sites per cell.

As used herein, the “biological activity of an urotensin II receptor” refers to an activity exerted by the urotensin II receptor as determined in vivo, or in vitro, according to standard techniques. Such an activity can be a direct activity such as the ability of an urotensin II receptor to bind to an urotensin II (U-II) or an analog thereof. The activity can be functional changes of cell physiology, such as calcium mobilization or changes in cellular electrical impedance. The biological activity of an urotensin II receptor can be an indirect activity, such as a signal transduction activity mediated by the urotensin II receptor via its interaction with one or more than one additional protein or other molecule(s), including but not limited to, interactions that occur in a multi-step, serial fashion. An urotensin II receptor has the biological activity of mediating the function of U-II or a functional equivalent thereof as an endothelium independent vasoconstrictor or an endothelium dependent vasodilator.

A “signal transduction” is the cascade of processes by which an extracellular signal interacts with a receptor at a cell surface, causing a change in the level of a second messenger, and ultimately effects a change in the cell function.

A “signal transduction activity mediated by urotensin II receptor” refers to a signal transduction, wherein the extracellular signal is urotensin II or a functional equivalent thereof. In one embodiment, a “signal transduction activity mediated by urotensin II receptor” is the cascade of processes by which urotensin II binds to an urotensin II receptor at a cell surface, causing a change in the level of a second messenger, such as calcium or cyclic AMP, and ultimately effects a change in the cell's function. The change in the cell's function can be the change of any cellular process urotensin II is involved in. Changes in the cell's function often lead to changes of the animal physiology. For example, a “signal transduction activity mediated by urotensin II receptor” can be an endothelium independent vasoconstriction or an endothelium dependent vasodilation triggered by urotensin II.

As described herein, a “test molecule”, “test compound”, or “candidate compound”, used interchangeably herein, each means a molecule that is subjected to the assay systems and methods described herein. Test compounds or candidate compounds encompass numerous chemical classes, although typically they are organic compounds. Preferably, they are small organic compounds, i.e., those having a molecular weight of more than 50 Kd yet less than about 2500 Kd. Candidate compounds comprise functional chemical groups necessary for structural interactions with polypeptides, and typically include at least an amine, carbonyl, hydroxyl or carboxyl group, preferably at least two of the functional chemical groups and more preferably at least three of the functional chemical groups. The candidate compounds can comprise cyclic carbon or heterocyclic structure and/or aromatic or polyaromatic structures substituted with one or more of the above-identified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound typically is a DNA or RNA molecule, although modified nucleic acids having non-natural bonds or subunits are also contemplated.

Candidate compounds can be obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Candidate compounds can also be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection (Lam (1997) Anticancer Drug Des. 12:145). Alternatively, libraries of natural compounds in the form of bacterial, fungal, plant and animal extracts are available or readily produced. Additionally, natural and synthetically produced libraries and compounds can be readily modified through conventional chemical, physical, and biochemical means.

Further, known pharmacological agents can be subjected to directed or random chemical modifications such as acylation, alkylation, esterification, amidation, etc. to produce structural analogs of the agents. Candidate compounds can be selected randomly or can be based on existing compounds that bind to and/or modulate the function of chloride channel activity. Therefore, a source of candidate agents is libraries of molecules based on a known compound that increases or decreases the biological activity of a U-II receptor, in which the structure of the known compound is changed at one or more positions of the molecule to contain more or fewer chemical moieties or different chemical moieties. The structural changes made to the molecules in creating the libraries of analog activators/inhibitors can be directed, random, or a combination of both directed and random substitutions and/or additions. One of ordinary skill in the art in the preparation of combinatorial libraries can readily prepare such libraries.

A variety of other reagents also can be included in the method. These include reagents such as salts, buffers, neutral proteins (e.g., albumin), detergents, etc. Other reagents that improve the efficiency of the assay such as nuclease inhibitors, antimicrobial agents, and the like can also be used.

Examples of methods for the synthesis of molecular libraries can be found in the art, for example in: Zuckermann et al. (1994). J Med. Chem. 37:2678. Libraries of compounds can be presented in solution (e.g., Houghten (1992) Biotechniques 13:412-421), or on beads (Lam (1991) Nature 354:82-84), chips (Fodor (1993) Nature 364:555-556), bacteria (U.S. Pat. No. 5,223,409), spores (U.S. Pat. No. 5,571,698), plasmids (Cull et al. (1992) Proc. Natl. Acad. Sci. USA 89:1865-1869) or phage (see e.g., Scott and Smith (1990) Science 249:386-390).

The term “high throughput” refers to an assay design that allows easy screening of multiple samples simultaneously, and provides a capacity for robotic manipulation. Another desired feature of high throughput assays is an assay design that is optimized to reduce reagent usage, or minimize the number of manipulations in order to achieve the analysis desired. Examples of high throughput assay formats include 96-well or 384-well plates, levitating droplets, and “lab on a chip” microchannel chips used for liquid handling experiments.

The present invention provides methods for identifying compounds that increase or decrease the biological activity of an U-II receptor. The inventive assay methods can be used to detect test compounds that increase or decrease the biological activity of an U-II receptor in any manner. Compounds that increase or decrease the biological activity of an U-II receptor can be compounds that interact directly with the U-II receptor in such a way as to affect the biological activity of U-II receptor. For example, such a compound can bind to the U-II receptor and affect the interaction of the receptor with U-II or other protein/molecule, such as a functional derivative of U-II, e.g., an U-II mimetic, or an agonist or antagonist of U-II. Compounds that increase or decrease the biological activity of an U-II receptor can also be compounds that interact indirectly with the U-II receptor in such a way as to affect the biological activity of U-II receptor. Such a compound can bind to protein(s) or molecules other than the U-II receptor, and affect the signal transduction activity of the U-II receptor. For example, such a compound can increase or decrease the amount or activity of a factor from the vascular endothelium, e.g., nitric oxide or prostacyclin, that is involved in the U-II receptor mediated vasodilation.

One general aspect of the invention is a method of identifying a cell having a functional urotensin receptor, comprising the steps of: 1) contacting a candidate cell with an effective amount of an urotensin II or a functional equivalent thereof; 2) measuring the electrical impedance of the candidate cell before and after the administration of the urotensin II or the functional equivalent thereof; and 3) identifying the candidate cell as having a functional urotensin receptor by an increase in the electrical impedance of the candidate cell upon the administration of the urotensin II or the functional equivalent thereof.

The method of the invention can be used to test a variety of candidate cells. Native cell lines can be tested for the presence of a functional urotensin II receptor. For example, RMS13, TE671, rat medullary thyroid cell line (6-23), human primary skeletal muscle cells (hSKMC), and any other of the 49 primate and rodent cell lines described in Douglas et al (Douglas, 2004, supra) can be tested by the method of the invention. Recombinant cells having a recombinant urotensin II receptor gene can also be tested for the proper expression of a functional urotensin receptor.

Another general aspect of the invention is a method of testing a candidate compound for its ability to increase the biological activity of an urotensin II receptor in a cell, comprising the steps of: (1) administering the candidate compound to the cell; (2) measuring the electrical impedance of the cell; and (3) correlating an increase in the electrical impedance of the cell with the ability of the candidate compound to increase the biological activity of the urotensin II receptor in the cell.

In another embodiment, the invention provides a method of testing a candidate compound for its ability to decrease the biological activity of an urotensin II receptor in a cell, comprising the steps of: (1) administering to the cell an effective amount of an agent that is known to increase the biological activity of an urotensin II receptor, wherein the effective amount of said agent induces an increase in the electrical impedance of the cell; (2) administering to the cell the candidate compound; (3) measuring the electrical impedance of the cell; and (4) correlating an inhibition of the agent-induced increase in the electrical impedance of the cell with the ability of the candidate compound to decrease the biological activity of the urotensin II receptor in the animal.

Any cell having a functional urotensin receptor can be used in the method of the invention. Such cells can be identified by the method of the invention described supra. In a particular embodiment, the cell can be RMS13. In other embodiment, the cell can be any of the subclones of RMS13 that have increased U-II binding sites and stronger calcium mobilization response upon U-II stimulation, such as 6D9, 4G5, and 10A7 (Minor et al., U.S. Application Ser. No. 60/708,221, filed Aug. 15, 2005). In yet another embodiment, the cell can be a cell that expresses U-II receptor recombinantly. The recombinant cell can have increased U-II receptor per cell or desired mutations on the promoter or coding sequence of the U-II receptor gene, etc. For example, as illustrated in the Examples, CHOrUII, a Chinese Hamster Cell (CHO) with a recombinant U-II receptor gene can be used in the invention.

In the method of identifying a compound that decreases the biological activity of an U-II receptor in a cell, the agent that is known to increase the biological activity of an urotensin II receptor can be U-II or a functional equivalent of U-II. The term “effective amount of an agent” as used in this aspect, means that amount of an agent that elicits a detectable increase in the electrical impedance of a cell having a functional U-II receptor when the agent is administered to the animal. It is readily appreciated that the effective amount can vary depending upon the particular compound used, the strength of the preparation, and the means of how to measure the electrical impedance, etc. In addition, factors associated with the particular cell used, including the type of cells, the number of U-II receptors per cell, and the culturing conditions for the cell, etc. can also result in changes of the “effective amount of an agent”. Methods are known in the art for determining the “effective amount of an agent”. For example, a skilled artisan can determine the range of the effective amount of an agent experimentally by testing the agent at various dosages with a certain cell, and measuring the electrical impedance of the cell at various time points after administration of the compound to the cell.

When U-II is used to induce an increase in electrical impedance of a cell of RMS13 or a subclone of RMS13 (6D9), as shown in the Example described infra, the EC₅₀ of U-II is about 2.7×10⁻¹⁰ M or 4.2×10⁻¹⁰ M, respectively. Therefore, in some embodiments, the “effective amount of U-II” to induce an increase in electrical impedance of a cell of RMS13 or a sub-clone thereof can be in the range of above about 0.1 nM.

As used herein, the phrase “an inhibition of the agent-induced increase in the electrical impedance of the cell” means prevention, blocking, prohibiting, decreasing, lowering, or abolishing of the agent-induced increase in the electrical impedance of the cell. Such an inhibition can be determined, for example, by comparing the electrical impedance of the cell after the administration of the test compound with that from the cell that is administered with the active agent alone, without the test compound.

In the method of identifying a compound that decreases the biological activity of the U-II receptor, agents that are known to activate or increase the biological activity of an U-II receptor can be administered to the cell prior to, concurrent with, or after the administration of the test compound to the cell.

The electrical impedance of a cell can be measured using any methods known to a person skilled in the art. For example, the electrical impedance can be measured using single frequency scanning mode or multi-frequency scanning mode. In preferred embodiments, α- and β-dispersions, which appear from 100 Hz to 10 KHz and from 100 KHz to 10 MHz, respectively, should both be considered in the measurement of the dielectric behavior of a cell (Gheorghiu, 1996, Bioelectromagnetics 17:475-482). (α-dispersion information enables the evaluation of the biological cell resting potential and cell morphology, while information on the permittivity and the conductivity of cellular subcompartments—for example the cell membrane, the cytoplasm—are revealed only in the β-dispersions range. Depending on the cell being used in the assay, one means of measurement can be preferred over another. A cell that has less change in electrical impedance upon U-II stimulation would require a more sensitive means of measurement.

In particular embodiments of the invention, the electrical impedance of a cell is measured using a label-free cell-based cellular dielectric spectroscopy (CDS) technology that has been described in WO 2005005979.

In an illustrative embodiment, CellKey™, a commercially available (MDS Sciex, Concord, Ontario), fully automated CDS system is used to measure the electrical impedance.

The methods of the invention can be combined with other means of testing a compound for its ability to increase or decrease the U-II biological activity. For example, a compound that increases or decreases the U-II biological activity can be first identified by its ability to bind to an U-II receptor, then tested for its ability to alter the electrical impedance of a cell. In another embodiment, a compound identified by its ability to alter the impedance of a cell using a method of the invention, can be further tested for its ability to alter another cellular function, such as calcium mobilization, i.e., using, e.g., a FLIPR assay, or vise versa. In yet another embodiment, a compound identified by its ability to alter the impedance of a cell using a method of the invention can be tested in an animal model for its ability to cause animal physiology changes. For example, it was observed that administering U-II to a rat induced an increase in the redness or skin temperature of the rat ear (Qi et al., U.S. Patent Application No. 60/680,449, filed May 12, 2005); systemic administration of human U-II to anethetized monkeys resulted in a decrease in total peripheral conductance and cardiac contractility (Ames, 1999, supra); intravenous bolus injection of U-II into anethetized rats produced a decrease in mean arterial pressure, left ventricular systolic pressure and cardiac contractility (Hassan, 2003, Can J Physiol Pharmacol 81(2): 125-8); bolus injection of U-II to conscious rats evokes an initial response consisting of tachycardia and hypotension, followed by a later phase (30-120 min post injection) of tachycardia and hypertension (Gardiner, 2004, Br J Pharmacol 143(3): 422-30); and indomethacin and L-NAME together prevented both phases of the haemodynamic responses to U-II (Gardiner, 2004, supra); in conscious rats the predominant hemodynamic effect of U-II is systemic vasodilatation with dose-dependent tachycardia (Gardiner, 2001 et al., Br. J. Pharmacol. 132(8): 1625-9; and Lin et al., 2003, J. Hypertens 21(1): 159-65).

This invention will be better understood by reference to the example that follows. Those skilled in the art will readily appreciate that the example is only illustrative of the invention and not limiting.

### EXAMPLES

### Example 1

Cells of RMS13 and 6D9 have endogeneous human U-II receptors. CHOrUII cells have recombinant rat U-II receptors. RMS13 was obtained from ATCC (ATCC NO: CRL-2061™, Manassas, Va.). 6D9 was a sub-clone of RMS13 that has increased U-II binding and calcium mobilization response upon U-II stimulation (Minor et al., U.S. Application Ser. No. 60/708,221, filed Aug. 15, 2005). The parent of the CHOrUII cells, CHO-K1 was obtained from ATCC (CAT# CRL-9618_CHOK1). The rat UT receptor was recombinantly cloned into CHO-K1 following standard molecular biology cloning techniques. The CHOrUII cells were under G418 selection. U-II was purchased from Sigma (cat#U-7257). Angiotensin was purchased from Sigma. Urantide (PUT-3639-P1) was purchased from Peptide International (Louisville, Ky.).

Cells of RMS13 and 6D9 were cultured in RPMI medium (ATCC 30-2001) containing 10% FBS (Cat. No. SH30070.03, Hyclone, Logan, Utah). CHOrUII cells were grown in DMEMF12 (Gibco #11330-032) medium containing G418 at 1 mg/ml concentration. Cells were plated at 60 or 70K/well in a medium indicated above overnight. The medium was removed and replaced with Hanks balanced salt solution (HBSS, Gibco) containing 20 mM Hepes and 0.1% BSA. Electrical impedance was measured 2-5 minutes prior to compound addition. Compounds were added and measurements continued. The electrical impedance of the cells was measured using CellKey™ (MDS Sciex, Concord, Ontario) according to the protocol of the manufacturer, see for example Ciambrone et al. (2004, J Biomol Screen. 9(6): 467-80). In particular, 100 mV voltage was applied to the cells with a sweeping frequency range of 1 kHz to 10 mHz.

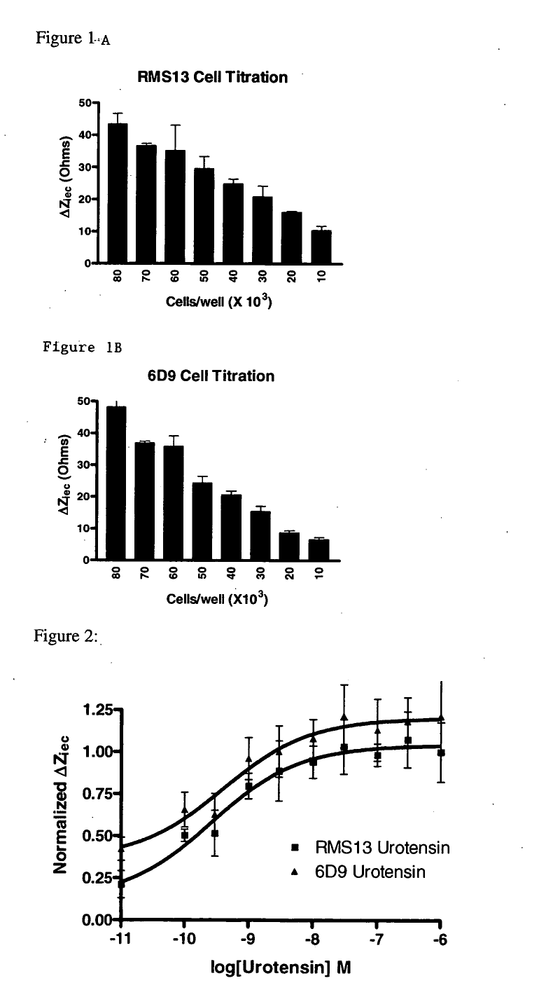

Cell density-dependence responses to Urotensin II (500 nM) were analyzed to determine the optimum cell density for the CDS assay at 28° C. A density of 70,000 cells/well was determined to be optimal for the RMS13 and 6D9 cells (FIGS. 1A and B), and 45,000 cells/well was determined to be optimal for the CHOrUII cells (data not shown).

Urantide was a recently discovered, competitive, potent (pA2=8.3) and pure UT receptor antagonist in rat aorta vasoconstriction assays (Patacchini, 2003, Br. J. Pharmacol. 140(7): 1155-8). It had minimal binding activity to proteins within a panel of receptor/ion channels (data not shown). A more recent report showed that urantide is a potent U-II agonist in CHO cells over-expressing the human UT receptor (Camarda, 2004, Eur. J. Pharmacol. 498(1-3): 83-6).

The effect of U-II, urantide, and angiotensin on a cell, particularly the electrical impedance of the cell was tested using the CDS device. Other U-II antagonists or inverse agonist can also be tested using similar protocols.

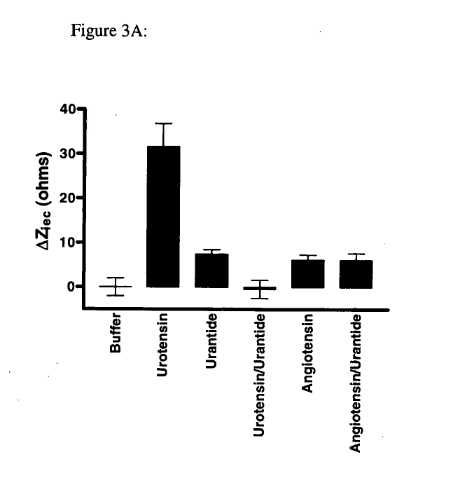

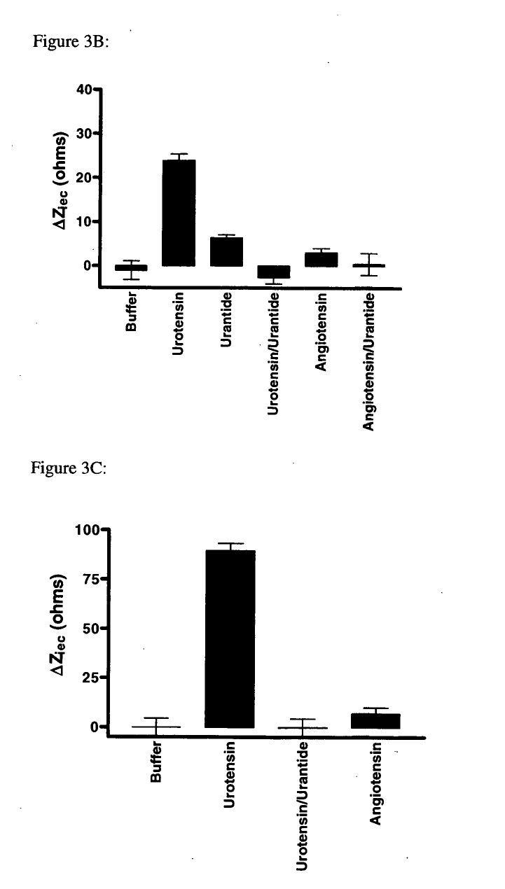

Administering U-II to the cells caused an increase of the electrical impedance of the RMS13 or 6D9 cells in a dose dependent manner (FIG. 2). Similar result was also observed with the CHOrUII. Urantide blocked the U-II stimulated increase, while angiotensin did not (FIGS. 3A, B, and C), suggesting that the change in the electrical impedance is specific to the activity of U-II. An increase in U-II activity results in an increase in the impedance; while a decrease in U-II activity results in a decrease in impedance. The result demonstrated that a compound that increases or decreases the biological activity of an U-II receptor can be identified by its ability to increase or decrease the electrical impediment of the cell.

### Example 2

The RT-CES™ system (ACEA Biosciences, Inc. San Diego, Calif.) was used to determine U-II effect on impedance. (Solly et al., Application of Real-Time Cell Electronic Sensing (RT-CES™) Technology to Cell-Based Assays. Assay and Drug Development Technologies. 2004; 2(4):363-372). The system is comprised of three components: an electronic sensor analyzer; a device station; and a 16 well strip. In operation, the device station, which holds the 16 well strip and which is capable of switching any one of the wells to the sensor analyzer for impedance measurement, with cells cultured in the wells is placed in an incubator. The electronic sensor analyzer automatically selects wells to be measured and continuously conducts measurements on the wells. The electrical impedance is transferred to a computer and plotted. In the ACEA system, a calculated parameter (cell index) is used as a measure of impedance. In this experiment, cells were plated at 40 thousand per well and allowed to incubate at 37° C. overnight. Following incubation, an initial baseline read was taken on the RT-CES™ followed by agonist addition and additional reads. The EC₅₀ for urotensin II generated using this system is comparable to that seen with the CellKey™ system.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A functional assay of the biological activity of an urotensin receptor in a cell comprising the step of measuring the electrical impedance of the cell.

2. A method of identifying a cell having a functional urotensin receptor, comprising the steps of:
a. contacting a candidate cell with an effective amount of an urotensin II or a functional equivalent thereof;
b. measuring the electrical impedance of the candidate cell before and after the administration of the urotensin II or the functional equivalent thereof; and
c. identifying the candidate cell as having a functional urotensin receptor by an increase in the electrical impedance of the candidate cell upon the administration of the urotensin II or the functional equivalent thereof.

3. The method of claim 2, wherein the electrical impedance of the candidate cell is measured using a cellular dielectric spectroscopy.

4. The method of claim 2, wherein the electrical impedance of the candidate cell is measured using CellKey™ (MDS Sciex, Concord, Ontario).

5. A method of testing a candidate compound for its ability to increase the biological activity of an urotensin II receptor in an cell, comprising the steps of:
a. administering the candidate compound to the cell;
b. measuring the electrical impedance of the cell; and
c. correlating an increase in the electrical impedance of the cell with the ability of the candidate compound to increase the biological activity of the urotensin II receptor in the cell.

6. The method of claim 5, wherein the electrical impedance of the cell is measured using a cellular dielectric spectroscopy.

7. The method of claim 5, wherein the electrical impedance of the cell is measured using CellKey™ (MDS Sciex, Concord, Ontario).

8. The method of claim 5, wherein the cell has an endogeneous urotensin receptor.

9. The method of claim 8, wherein the cell is RMS13 (ATCC® Number: CRL-2061™).

10. The method of claim 8, wherein the cell is a subclone of RMS13 having increased urotensin binding sites per cell.

11. The method of claim 10, wherein the cell is 6D9, 4G5, or 10A7.

12. The method of claim 5, wherein the cell has a recombinant urotensin receptor.

13. A method of testing a candidate compound for its ability to decrease the biological activity of an urotensin II receptor in a cell, comprising the steps of:
a. administering to the cell an effective amount of an agent that is known to increase the biological activity of an urotensin II receptor, wherein the effective amount of said agent induces an increase in the electrical impedance of the cell;
b. administering to the cell the candidate compound;
c. measuring the electrical impedance of the cell; and
d. correlating an inhibition of the agent-induced increase in the electrical impedance of the cell with the ability of the candidate compound to decrease the biological activity of the urotensin II receptor in the cell.

14. The method of claim 13, wherein the electrical impedance of the cell is measured using a cellular dielectric spectroscopy.

15. The method of claim 13, wherein the electrical impedance of the cell is measured using CellKey™ (MDS Sciex, Concord, Ontario).

16. The method of claim 13, wherein the cell has an endogeneous urotensin receptor.

17. The method of claim 16, wherein the cell is RMS13 (ATCC® Number: CRL-2061™).

18. The method of claim 16, wherein the cell is a subclone of RMS13 with increased urotensin binding sites per cell.

19. The method of claim 18, wherein the cell is 6D9, 4G5, or 10A7.

20. The method of claim 13, wherein the cell has a recombinant urotensin receptor.

21. The method of claim 13, wherein the agent that is known to increase the biological activity of the urotensin II receptor is an urotensin II or a functional equivalent thereof.

22. The method of claim 13, further comprising a step of testing the candidate compound for its ability to bind to an urotensin II receptor.

23. The method of claim 13, further comprising a step of testing the candidate compound for its ability to elicit an urotensin II receptor mediated change in calcium mobilization of the cell.

24. The method of claim 13, further comprising a step of testing the candidate compound for its ability to elicit an urotensin II receptor mediated change in animal physiology of an animal.

25. The method of claim 24, further comprising the step of monitoring the redness of skin temperature of the ear of a rat.

26. The method of claim 2, wherein the electrical impedence of the candidate cell is measured using RT-CES.


SEQUENCE LISTING (10 sequences)

SEQ ID NO: 1 - protein, 6 aa, Artificial Sequence
  Description of Artificial Sequence Synthetic peptide
CFWKYC

SEQ ID NO: 2 - protein, 11 aa, Homo sapiens
ETPDCFWKYCV

SEQ ID NO: 3 - protein, 13 aa, Unknown Organism
  Description of Unknown Organism Frog sequence
AGNLSECFWKYCV

SEQ ID NO: 4 - protein, 12 aa, Unknown Organism
  Description of Unknown Organism Trout sequence
GGNSECFWKYCV

SEQ ID NO: 5 - protein, 12 aa, Cyprinus sp.
GGGAECFWKYCV

SEQ ID NO: 6 - protein, 12 aa, Sus sp.
GTPSECFWKYCV

SEQ ID NO: 7 - protein, 12 aa, Sus sp.
GPPSECFWKYCV

SEQ ID NO: 8 - protein, 13 aa, Rattus sp.
HGTAPECFWKYCI

SEQ ID NO: 9 - protein, 13 aa, Mus sp.
HGAAPECFWKYCI

SEQ ID NO: 10 - protein, 8 aa, Artificial Sequence
  Description of Artificial Sequence Synthetic peptide
ACFWKYCV